(19) United States
(12) Patent Application Publication
(10) Pub. No.: US 2006/0161230 A1
(43) Pub. Date: Jul. 20, 2006

(54) Method of applying thermodynamic therapy for the rejuvenation of a subject

(75) Inventor: John P. Craven, Honolulu, HI (US)

Correspondence Address: KRAMER & AMADO, P.C.;Suite 240, 1725 Duke Street, Alexandria, VA, 22314, US

(73) Assignee: COMMON HERITAGE CORPORATION, Honolulu, HI (US)

(21) Appl. No.: 11/036,056

(22) Filed: Jan. 18, 2005

(51) Int. Cl. A61F 7/00

(52) U.S. Cl. 607/104

(57) ABSTRACT

The present invention relates to a method for treating a subject using thermodynamic therapy through the application of cold water to the subject. The therapy also includes a method for improving the metabolism of the subject and inducing paradoxical sleep in the subject.

### BACKGROUND OF THE INVENTION

1. Field of the Invention

This invention relates generally to a method of treatment for the body of the subject wherein the subject is treated with thermodynamic therapy.

2. Description of Related Art

Thermogenesis is the process by which the body creates heat metabolically through the burning of stored energy. This process has been observed in a number of biological situations, such as animal hibernation, cold adaptation and fat reduction. While the results of these metabolic processes are evidenced on a systemic level, the roots of internally generated metabolic heat are cellular in nature. In particular, the interaction between the body's individual cells and the cellular power plants or mitochondria form the basis for the therapeutic effects of thermogenesis.

It has been concluded through experimentation that the electrons that flow into the mitochondria must be transported through a thermionic or thermoelectric process that is based upon the difference in temperature between the cytoplasm surrounding the mitochondria. Taking this transport into account, there must be a mechanism that converts incoherent energy to ordered energy and, in particular, generates high energy electrons which attach themselves to the ATP (Adenosine Triphosphate) molecules as they exit the mitochondria. Empirical evidence has led to the theory that the efficiency of a cell engaged in the metabolic process, during the aerobic phase, is a function of the difference in temperature between an interior of the mitochondria of a cell and the temperature of the cytoplasm surrounding the mitochondria, and the temperature difference between the exothermic chemistry and the cytoplasm. During the anaerobic phase, heat is generated from the blood and the exothermic process in the mitochondria. Relatively cold temperatures exist in the skin cells of the subject due to the temperature of the ambient region. These conditions produce a temperature gradient across the cell structure that enables a diffusion process to transport the relatively lower temperatures to the interior of the cell to effectively remove the heat generated from the blood and the exothermic process. Indeed, in a young, healthy person, the basic body configuration will optimize this process, provided that the circulatory process carries an adequate amount of oxygen to the cell. The result of younger people having the ability to efficiently reject heat from the interior of the cell may be seen after a young person has engaged in vigorous and very physical activity.

During rigorous activity and during physical exercise, such as running for example, heat is generated in the joints. This heat produced drives the oxygen molecules that are attached to an iron ion on each of four hemes on each hemoglobin molecule. These free oxygen molecules are deleterious and will oxidize the tissue over the course of the activity. As the blood traveling through the person's circulatory system passes the knee, for example, oxygen will be taken from the surrounding muscles instead of the cells surrounding the knee itself, as those cells are depleted of oxygen. The muscles in turn will start to release lactic acid that causes the familiar lactic acid burn that runners and weight lifters experience during the duration of the activity. When the exercise has been completed, the cells and the muscles involved in the activity will have to repair themselves using the normal mechanisms of healing. The empirical evidence gathered has suggested that elderly people do not have the metabolic efficiency to effect this repair, as do younger people who have performed the same activity or activities. Indeed, many elderly people who have been exposed to rigorous activity over a period of time suffer from atrophy that may even become permanent depending upon the circumstances. This phenomenon helps to explain why it only takes a few hours to a day for a younger person to recover from such activity as compared to a recovery lasting a few days to longer periods of time for an elderly person engaged in the same or a similar activity. Moreover, the inability of older people to reject heat may be a principle cause of the aging phenomenon.

Further evidence suggests that as a person engages in rigorous exercise, the oxidation process that the body undergoes for the regeneration of ATP can no longer produce enough ATP to keep up with the energy demands placed upon the body. As the oxygen supplies are depleted, the cell shifts to an aerobic mode similar to fermentation in plants. The aerobic phase of respiration takes place in the cytoplasm of the cell and requires the presence of a sugar called glycogen and an enzyme named creatine. Glycogen is manufactured from the surplus energy that is generated during muscular activity. During the aerobic phase, two ATP molecules per mole are required to break down the glycogen into 4 ATP and two moles of pyrhuvic acid. The pyrhuvic acid is in turn available as fuel for the mitochondria that will generate 40 ATP, if the oxygen is available. Thus, a sustained athletic performance can be maintained as long as there are reserves of glycogen. This effect also helps to explain why young athletes who have efficient metabolic processes can recover in a day or two while older persons with inefficient metabolism will take weeks or months to restore their ATP supplies.

An exothermic reaction in the mitochondria is produced when oxygen, transported by the blood stream, is introduced into the mitochondria and oxidizes a fuel that is also introduced into the mitochondria. This fuel is either a carbohydrate, which has been ingested, or one of a number of acids, such as lactic acid or pyrhuvic acid, generated by other metabolic processes. The major function of the mitochondria is to convert ADP (Adenosine Diphosphate), which is the depleted carrier of the body's metabolic energy, to ATP (Adenosine Triphosphate). ATP is the carrier of the body's metabolic energy provided that the electrons in the phosphate are “hot electrons”; i.e. in the outer shells of the Phosphorus atoms. During this energy-producing phase, a process called phosphorilization occurs wherein a stream of high-energy electrons are transported through the mitochondria until they attach to the appropriate chemical bond. Those having ordinary skill in the art will have a clear understanding of this cellular energy conversion process and the associated aerobic and anaerobic phases of energy production. However, the transportation mechanism that allows the electrons to flow into the mitochondria has not been adequately explained. Moreover, the benefits of utilizing this transport mechanism have not been realized in conventional cold temperature therapies.

Therefore, there is a need for a safe and effective method for inducing internal thermogenesis. As seen in nature, animal adaptation to cold results in a degree of thermogenesis. However, this thermogenesis is not targeted to maximize the beneficial effects of the metabolic processes while limiting the deleterious effects of cold on the body. Furthermore, application of cold may be specifically targeted to create other therapeutic effects outside of metabolically generated heat. Therefore, it is an object of this invention to apply cold to a subject in a manner that is therapeutically beneficial to the subject without resulting in deleterious side effects.

The foregoing objects and advantages of the invention are illustrative of those that can be achieved by the present invention and are not intended to be exhaustive or limiting of the possible advantages which can be realized. Thus, these and other objects and advantages of the invention will be apparent from the description herein or can be learned from practicing the invention, both as embodied herein or as modified in view of any variation which may be apparent to those skilled in the art. Accordingly, the present invention resides in the novel methods, arrangements, combinations and improvements herein shown and described.

### SUMMARY OF THE INVENTION

In light of the present need for a thermodynamic therapy for the rejuvenation of the body, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

The present invention includes a method for treating a subject through thermodynamic therapy comprising: applying cold water to regions of the subject's body; wherein the regions of the subject's body are regions that store adipose tissue; applying cold water to selected body parts of the subject; wherein the selected body parts contain elements of the lymphatic system; and insulating the subject's body from atmospheric heat.

The present invention also includes a method for improving the metabolism of a subject comprising: lowering the external body temperature of a subject; wherein the subject's external body temperature is lowered through application of cold water to the exterior of the subject; insulating the body of the subject from atmospheric heat; and returning the subject's body to normal external body temperature through metabolically generated heat.

The present invention further includes a method for inducing paradoxical sleep in a subject comprising: applying cold water to body of the subject; wherein cold water is applied to regions of the subject's body that store adipose tissue; wherein cold water is applied to parts of the subject's body that contain elements of the lymphatic system; and allowing the subject to begin a period of sleep.

### DETAILED DESCRIPTION OF THE PREFERRED EMBODIMENTS OF THE INVENTION

The present invention is a method for treating a subject through thermodynamic therapy. Thermodynamic therapy of a subject involves the modification and manipulation of internal and external heat formed as a result of the internal processes of the body. The internal and external temperatures of the body are altered by thermal modification of selected areas of flesh adjacent to the skin. This thermal modification results in a temperature difference between the mitochondria and the cytoplasm. The thermodynamic therapy further results in the improvement of metabolic processes; the initiation of paradoxical sleep; the growth and repair of body tissue; and the treatment of various body ailments, such as skin irregularities, inflammation, joint pain, muscle pain, bone pain, excessive fat deposit, vascular edema and decreased muscle tone.

The therapeutic method of the present invention is primarily achieved through the application of cold water to the body of a subject. In a preferred embodiment, the cold water temperature is between 1° C. and 10° C. The cold water is preferably kept above the temperature of freezing to prevent tissue injury to the subject. In another preferred embodiment, the cold water temperature is equal to or below 7° C., preferably between 5° C. to 7° C.

In a preferred embodiment of the invention, the therapeutic method is performed by applying cold water to specific areas of the body of the subject. The cold water may be applied directly to the body or indirectly where the cold water is contained in a thermally conductive container that is placed against the body. Direct application may include application of cold water to the body with applicators that may retain water including, but not limited to, towels, wash cloths, sponges, brushes or other devices known in the art. Direct application may also include, but is not limited to, pouring, spraying, inundating or immersing the specific areas of the body with cold water.

The specific areas of the body treated with cold water are those areas which are utilized in the thermodynamic therapy. In a preferred embodiment of the invention, the specific areas of the body include specific regions where adipose tissue is stored or concentrated. In another preferred embodiment of the invention, the specific regions of the body that store adipose tissue may include, but are not limited to, the belly, torso, midriff, flank, groin, buttocks, thighs and combinations thereof. In another preferred embodiment of the invention, the specific areas of the body include body parts that contain elements of the lymphatic system. In a preferred embodiment of the invention, elements of the lymphatic system are defined as lymph nodes, lymph ducts and entrances to lymph ducts. In another preferred embodiment of the invention, the body parts that contain elements of the lymphatic system may include, but are not limited to, the hands, feet, face, neck, breast and combinations thereof. The application of cold water may also be used to treat body ailments that are localized in specific areas of the body. In a preferred embodiment, these specific areas may include the ankles, knees, elbows, back, neck, limbs, joints, adipose deposits, inflamed tissue, skin irregularities and combinations thereof. In a preferred embodiment of the invention, the localized application of cold water may be directed to alleviate individual joint pain, muscle aches or damage, areas of excessive fat deposit, inflamed body organs or tissue, inflamed regions of skin, localized skin irregularities such as liver spots, blemishes, contusions, scars, varicose veins, acne, cellulite and other related ailments. Cold water may be applied to the body in the specific areas until the flesh located at the specific areas becomes cold to the touch and pink in color.

To facilitate a more efficient application of the cold water to the subject's body, the therapy may include the subject wearing a specialized suit. In a preferred embodiment of the invention, the subject may wear a thermally insulated suit modified to accept application of cold water. The suit may cover the entire body of the subject and is modified where the areas designated for cold water application are bare. In a preferred embodiment of the invention, the areas of the suit that cover the hands, feet, midriff, torso, groin, breast, face and neck may be removed. More particularly the breast area encompassing the thoracic region and the thymus may be bare. In another preferred embodiment, other areas of the body may be selectively bared in order to perform localized treatment. The suit may include several openings sealed by any openable sealing means known in the art, such as buttons, zippers, zip-lock, straps, ties or hook and loop, where specific areas of the body may be accessed through these openings. Such openings may include, but are not limited to elbow or knee flaps. In a preferred embodiment where the suit is used, the subject may wear the suit and be fully immersed in a body of cold water large enough to contain the entire subject.

Following application of cold water to the body of a subject, the therapy may include forcing the body to generate internal heat in order to warm the specific areas treated with cold water. The body will generally seek to replace the heat lost in the external areas that were chilled by the cold water. In a preferred embodiment of the invention, the subject is insulated from atmospheric temperature, where the subject's body cannot absorb heat from the atmosphere. Since the body cannot absorb heat from the atmosphere, it must generate the heat internally through the metabolic processes. In a preferred embodiment of the invention, the subject's body is insulated through the use of an insulating body wrap, covering, blanket, sheet or suit.

The thermodynamic therapy may also induce the patient to enter into paradoxical or REM sleep. Paradoxical or REM sleep is the time period in which the body suppresses certain metabolic functions such as digestion, muscle tonicity and mechanical movement in favor of growth, replacement and repair of body cells and tissue and elimination of unwanted fat deposits. Additionally, paradoxical sleep is the time period during which the brain rejuvenates and is required for the subject to feel fully rested and refreshed upon waking. Paradoxical sleep is induced where cooling of the cytoplasm of a significant number of body cells reduces and suppresses the output of ATDP. Thermodynamic therapy accomplishes this by cooling selective body areas and requiring the body to generate metabolic heat in other areas to warm the cooled areas. The metabolic heat is measured as coherent energy. The coherent energy effects the transition from normal sleep to paradoxical sleep. Therefore, in a preferred embodiment of the invention, following the application of cold water to the body of the subject, the subject is permitted to assume a position from which the subject may enter into paradoxical sleep. The position is one in which the subject feels comfortable sleeping, such as the supine, prone, fetal or any other lying position. The subject may or may not be insulated from atmospheric heat during the period of paradoxical sleep. In a preferred embodiment of the invention, the subject is insulated form atmospheric heat before entering into paradoxical sleep.

Prior to the application of cold water to the body of the subject, the subject may be prepared for the thermodynamic therapy. In a preferred embodiment of the invention, the subject may undertake a preparation regimen before entering into the thermodynamic therapy. In another preferred embodiment of the invention, the preparation for thermodynamic therapy may include a sleep regimen. Before the subject enters therapy, the subject may sleep for a limited amount of time. In a preferred embodiment of the invention, the limited amount of time may be greater than one hour but less than eight hours. In another preferred embodiment of the invention, the subject may fall asleep at their normal sleeping time and be woken for therapy at four hours before their regular waking time. The four hours of normal sleep may be replaced by four hours of paradoxical sleep induced by the thermodynamic therapy. The subject may undertake the thermodynamic treatment at any time during the day and may not require paradoxical sleep or sleep prior to the treatment.

In another preferred embodiment of the invention, the preparation regimen of the subject includes a thermal preparation regimen. The thermal preparation regimen may include application of both heat and cold. The application of heat may be administered through a hot shower, hot tube, sauna or other method of heating the body to provide a uniform internal thermal environment. The application of cold may follow the application of heat by a cold shower, cold water immersion, cold air immersion or other method of cooling to provide a uniform skin temperature and uniform difference in temperature over the entire body.

In another preferred embodiment of the invention, the preparation for thermodynamic therapy may include an exercise regimen for the subject. The exercise regimen may entail any light exercise known in the art. Any light exercise may be used to prepare for the thermodynamic therapy, where the light exercise discharges excess quantity of ATP molecules and converts them to ADP.

In another preferred embodiment of the invention, the preparation for thermodynamic therapy may include a nutritional regimen for the subject. In a preferred embodiment of the invention, the therapy takes place after the subject has fully digested their regular meals and completed any evacuatory functions. In another preferred embodiment of the invention, the subject ingests a specific nutritional regimen designed for use in the thermodynamic therapy. The nutritional composition may include the vitamins, minerals and nutrients required by the body to repair and create new cells and tissue. In a preferred embodiment of the invention, the vitamins, minerals, and nutrients may include, but are not limited to, phosphorus, potassium, vitamin C, iron, magnesium, zinc, calcium, collage, protein, carbohydrates and combinations thereof. In another preferred embodiment of the invention, any combination of nutritional supplementation is useful for the preparatory regimen where the combination contains more than 25% of the minimum recommended daily requirement of the elements required for the creation and repair of cells, especially phosphorous.

The therapy itself may be administered at least once a day. Additionally, the therapy may be repeated in succession especially where more than four hours of paradoxical sleep is needed.

The following is a detailed Example of the method of treating a subject through thermodynamic therapy as described above. This example is only meant to illustrate a possible procedure for the application of the method of the invention.

### EXAMPLE

In this example the therapy is administered once a day and may be used as a substitute for four hours of normal sleep. The subject retires for a period of light sleep of at least two hours or longer. The subject is then wakened at a time of about four hours before the subject's usual waking time. Before the therapy is administered, all of the digestion and elimination processes should be completed so that the body will be free to concentrate on the desired metabolic processes. At the start of each session the subject may take a hot shower or spend time in a sauna to provide a uniform internal thermal environment. The application of heat is followed by a cold shower to provide a uniform skin temperature and uniform difference in temperature over the entire body.

The applicant may also prepare through a light exercise and nutritional regimen. The light exercise discharges excess quantities of ATP and may include such light exercise as sit-ups, push-ups, jogging in place or other similar routines. The subject may then ingest a low calorie menu of nutrients containing the vitamins, minerals and nutrients that are required for the creation and repair of cells and tissues. A sample diet may include milk with ovaltine and/or molasses (for phosphorus and potassium), orange juice (for vitamin C), raisins (for iron), almonds (for magnesium, zinc and calcium) and gelatin (for collage). Other dietary supplements may include protein bars or other similar multi-nutrient sources.

Following the preparation regimen, the subject may be submitted to application of cold water. The cold water is initially applied to regions of the body that store adipose tissue. At the same time the rest of the body must maintain body temperature. A wash cloth or sponge is soaked in cold water (temperature of 5° C.-7° C.) and used to apply the cold water to the belly and torso. The process is repeated until the flesh is pink and the applying device does not absorb heat from the subject's body. If there are specific areas of the body other than those regions for storing adipose tissue that require localized treatment, these areas are also treated with cold water at this time.

Following the application of cold water to the regions of the body for storing adipose tissue, selected body parts of the subject are immersed in cold water. Specifically, the hands, feet, face, and flesh exterior to the lymph nodes, the entrances to the right lymphatic duct, and the Thoracic duct, or alternatively, left lymphatic duct and the Thymus, are immersed in cold water to induce the flow of blood to perfuse the peripheral capillary system that surrounds the entrances to the lymphatic system. Again, these areas are treated until the skin becomes pink and the underlying flesh is firm to the touch. The immersion may be followed by a final application of cold water to the torso.

Alternatively, if the subject is wearing a specialized suit for thermodynamic therapy, the subject may be fully immersed throughout the entire application of cold water. The specialized thermal suit will insulate the covered areas and allow the areas of bare skin to be chilled by the cold water. The bare areas of the suit may include the hands, feet, midriff, groin, breast area around the thymus, face and neck. The reproductive organs may be protected by a bikini bottom. Additionally, the suit may have sealable openings to allow access to other localized body areas for cold water application such as the elbows or knees. The suited subject may then be immersed in a tank or other large container of cold water until the skin becomes pink and the flesh is firmed.

Following application of cold water, the subject is insulated against atmospheric heat. In this example, the subject is wrapped in an insulated sheet. Once the subject is insulated, all heat required to warm the chilled areas must be generated metabolically.

Following insulation, the subject lies down on a bed, cot or mattress and begins to sleep. Soon after falling asleep, the subject will transition from normal sleep to paradoxical sleep. This may be evidence by the subject curling up into the fetal position or by the rapid eye movements of the subject signifying REM sleep. The subject will then spend the next two to six hours in paradoxical sleep after which the subject will awake fully energized and refreshed.

The process may be repeated immediately if it is desired to extend the therapy for another four hours. If the therapy includes paradoxical sleep and takes place during daylight, the subject should wear an eyeshield to prevent the eyeball from exposure to photons which will break the sleep mode.

Although the present invention has been described in detail with particular reference to preferred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

1. A method for treating a subject through thermodynamic therapy comprising:
applying cold water to regions of the subject's body; wherein the regions of the subject's body are regions that store adipose tissue;
applying cold water to selected body parts of the subject; wherein the selected body parts contain elements of the lymphatic system;
and insulating the subject's body from atmospheric heat.

2. The method of claim 1 further comprising preparing the subject's body for cold water application.

3. The method of claim 2 further comprising providing a uniform internal temperature for the subject.

4. The method of claim 2 further comprising providing a uniform skin temperature for the subject.

5. The method of claim 2 further comprising providing a uniform difference in temperature over the entire body.

6. The method of claim 2 further comprising administering a exercise regimen to the subject.

7. The method of claim 2 further comprising administering a nutritional regimen to the subject.

8. The method of claim 1 wherein the cold water temperature is 1° C.-10° C.

9. The method of claim 1 wherein the regions that store adipose tissue comprise the belly, torso, midriff, flank, groin, buttocks, thighs and combinations thereof.

10. The method of claim 1 further comprising applying cold water to areas of the body that require specific therapy.

11. The method of claim 10 wherein the areas that of the body that require specific therapy comprise the ankles, knees, elbows, back, neck, limbs, joints, adipose deposits, inflamed tissue, skin irregularities and combinations thereof.

12. The method of claim 1 wherein the selected body parts containing elements of the lymphatic system comprise body parts containing lymph nodes.

13. The method of claim 1 wherein the selected body parts containing elements of the lymphatic system comprise the hands, feet, face, neck, breast and combinations thereof.

14. The method of claim 1 further comprising insulating the subject from atmospheric heat.

15. The method of claim 14 wherein the subject is insulated by wrapping the subject in an insulating material.

16. The method of claim 1 wherein the subject generates metabolic heat to warm the regions to which cold water has been applied.

17. The method of claim 1 wherein the subject generates metabolic heat to warm the body parts to which cold water has been applied.

18. The method of claim 1 wherein the subject enters into paradoxical sleep.

19. The method of claim 1 wherein the subject wears a suit for facilitating thermodynamic therapy.

19. The method of claim 19 wherein the suit is thermally insulated.

20. The method of claim 19 wherein the suit comprises access points for applying cold water to the regions of the subject's body.

21. The method of claim 20 wherein the suit comprises access points for applying cold water to the body parts of the subject.

22. The method of claim 19 wherein a subject wearing the suit is immersed in cold water.

23. The method of claim 19 wherein the suit is removed after the application of cold water.

24. The method of claim 23 further comprising insulating the subject form atmospheric heat.

25. A method for improving the metabolism of a subject comprising:
lowering the external body temperature of a subject; wherein the subject's external body temperature is lowered through application of cold water to the exterior of the subject;
insulating the body of the subject from atmospheric heat; and
returning the subject's body to normal external body temperature through metabolically generated heat.

26. The method of claim 25 further comprising preparing the subject for the application of cold water.

27. The method of claim 26 further comprising providing a uniform internal temperature for the subject.

28. The method of claim 26 further comprising providing a uniform skin temperature for the subject.

29. The method of claim 26 further comprising providing a uniform difference in temperature over the entire body.

30. The method of claim 26 further comprising administering a exercise regimen to the subject.

31. The method of claim 26 further comprising administering a nutritional regimen to the subject.

32. The method of claim 25 wherein the cold water temperature is 1° C.-10° C.

33. The method of claim 25 wherein the cold water is applied to regions of the subject's body that store adipose tissue

34. The method of claim 33 wherein the regions that stores adipose tissue comprise the belly, torso, midriff, flank, groin, buttocks, thighs and combinations thereof.

35. The method of claim 25 further comprising applying cold water to areas of the body that require specific therapy.

36. The method of claim 35 wherein the areas that of the body that requires specific therapy comprise the ankles, knees, elbows, back, neck, limbs, joints, adipose deposits, inflamed tissue, skin irregularities and combinations thereof.

37. The method of claim 25 wherein the cold water is applied to selected body parts of the subject that contain elements of the lymphatic system.

38. The method of claim 37 wherein the selected body parts containing elements of the lymphatic system comprise body parts containing lymph nodes.

39. The method of claim 37 wherein the selected body part containing elements of the lymphatic system comprises the hands, feet, face, neck, breast and combinations thereof.

40. The method of claim 25 wherein the subject is insulated by wrapping the subject in an insulating material.

41. The method of claim 25 wherein the subject enters into paradoxical sleep.

42. The method of claim 25 wherein the subject wears a suit for facilitating the application of cold water to the body of the subject.

42. The method of claim 42 wherein the suit is thermally insulated.

43. The method of claim 42 wherein the suit comprises access points for applying cold water to the exterior of the subject's body.

44. The method of claim 42 wherein a subject wearing the suit is immersed in cold water.

45. The method of claim 42 wherein the suit is removed after subject's external body temperature is lowered.

46. The method of claim 45 further comprising insulating the subject form atmospheric heat.

47. A method for inducing paradoxical sleep in a subject comprising:
applying cold water to body of the subject;
wherein cold water is applied to regions of the subject's body that store adipose tissue
wherein cold water is applied to parts of the subject's body that contain elements of
the lymphatic system; and
allowing the subject to begin a period of sleep.

48. The method of claim 47 further comprising preparing the subject's body for application of cold water.

49. The method of claim 48 further comprising providing a uniform internal temperature for the subject.

50. The method of claim 48 further comprising providing a uniform skin temperature for the subject.

51. The method of claim 48 further comprising providing a uniform difference in temperature over the entire body.

52. The method of claim 48 further comprising administering a exercise regimen to the subject.

53. The method of claim 48 further comprising administering a nutritional regimen to the subject.

54. The method of claim 47 wherein the cold water temperature is 1° C.-10° C.

55. The method of claim 47 wherein the regions that store adipose tissue comprise the belly, torso, midriff, flank, groin, buttocks, thighs and combinations thereof.

56. The method of claim 47 further comprising applying cold water to areas of the body that require specific therapy.

57. The method of claim 56 wherein the areas that of the body that requires specific therapy comprise the ankles, knees, elbows, back, neck, limbs, joints, adipose deposits, inflamed tissue, skin irregularities and combinations thereof.

58. The method of claim 47 wherein the selected body parts containing elements of the lymphatic system comprise body parts containing lymph nodes.

59. The method of claim 47 wherein the selected body parts containing elements of the lymphatic system comprise the hands, feet, face, neck, breast and combinations thereof.

60. The method of claim 47 further comprising insulating the subject from atmospheric heat.

61. The method of claim 60 wherein the subject is insulated by wrapping the subject in an insulating material.

62. The method of claim 47 wherein the subject generates metabolic heat to warm the regions to which cold water has been applied.

63. The method of claim 47 wherein the subject generates metabolic heat to warm the body parts to which cold water has been applied.

64. The method of claim 47 wherein the subject wears a suit for facilitating applying cold water the subject's body.

65. The method of claim 64 wherein the suit is thermally insulated.

66. The method of claim 64 wherein the suit comprises access points for applying cold water to the subject's body.

67. The method of claim 64 wherein a subject wearing the suit is immersed in cold water.

68. The method of claim 64 wherein the suit is removed after the application of the cold water.

69. The method of claim 68 further comprising insulating the subject form atmospheric heat.
